(19) United States
(12) Patent Application Publication
(10) Pub. No.: US 2012/0329760 A1
(43) Pub. Date: Dec. 27, 2012

(54) RNASE 7 AS A MARKER FOR INFECTION

(75) Inventors: David Hains, Columbus, OH (US); Andrew Lawrence Schwaderer, Blacklick, OH (US); John David Spenser, Columbus, OH (US)

(73) Assignee: NATIONWIDE CHILDREN'S HOSPITAL, INC., Columbus, OH (US)

(21) Appl. No.: 13/450,662

(22) Filed: Apr. 19, 2012

Related U.S. Application Data
(60) Provisional application No. 61/477,157, filed on Apr. 19, 2011.

(51) Int. Cl. G01N 33/573; A61K 31/545; A61P 13/02; A61K 31/43; A61K 31/65; A61P 31/00; A61K 31/505; A61K 31/4178

(52) U.S. Cl. 514/152; 435/7.4; 514/275; 514/200; 514/390; 514/197

(57) ABSTRACT

Methods of identifying a subject having a urinary tract infection using RNase 7 are described. The methods include providing a test sample of urine from the subject, determining the level of RNase 7 in the test sample, and then comparing the level of RNase 7 in the test sample to the level of RNase 7 in a control. A higher level of RNase 7 in the test sample indicates that the subject has an acute urinary tract infection, whereas a lower level of RNase 7 in the test sample indicates that the subject only has a chronic urinary tract infection.

### CROSS-REFERENCE TO RELATED APPLICATIONS

This application claims priority to U.S. Provisional Patent Application No. 61/477,157, filed Apr. 19, 2011, which is hereby incorporated by reference in its entirety.

### STATEMENT REGARDING FEDERALLY SPONSORED RESEARCH OR DEVELOPMENT

This work was supported, at least in part, by National Institute of Health Grant No. 1RC4DK090937-01. The United States government has certain rights in this invention.

### BACKGROUND

The urinary tract, except for the urethral meatus, is usually sterile despite its proximity with fecal flora. The precise mechanism by which the urinary tract maintains sterility is not well understood. Recently, antimicrobial peptides (AMPs) have been shown to have an important role in innate immunity. Zasloff M., J Am Soc Nephrol; 18: 2810-2816 (2007). AMPs are a ubiquitous component of innate immunity produced by epithelial cells or hematopoietic cells. AMPs are mainly cationic proteins that possess antimicrobial activity against bacteria, enveloped viruses, fungi, and some protozoa. They may be constitutively expressed and/or induced by invading pathogens. Zaiou M., J Mol Med; 85: 317-329 (2007). Although many AMPs have been described in other organ systems, only a few AMPs have been studied in the human urinary tract. Ali et al., The Journal of urology; 182: 21-28 (2009).

Harder and Schröder first identified ribonuclease 7 (RNase 7) as an abundant protein in the human epidermis while examining protein extracts of normal skin for antimicrobial activity. Harder J, Schroder J M., J.B.C.; 277: 46779-46784 (2002). Subsequent studies demonstrated that RNase 7 is expressed in other organs, including the liver, gastrointestinal tract, heart, skeletal muscle, and respiratory tract. RNase 7 expression was noted in the kidney, although the extent of its expression and precise location were not characterized. Zhang et al., Nucleic acids research; 31: 602-607 (2003). Although the mechanisms for RNase 7's antimicrobial properties are not completely understood, its bactericidal activity has been linked to its capacity to permeate and disrupt the bacterial membrane, independent of its ribonuclease activity. Dyer et al., Mol Divers; 10: 585-597 (2006). Consequently, RNase 7 has potent antimicrobial activity against gram-negative bacteria, gram-positive positive bacteria, and yeast. Torrent et al., FEBS J 277: 1713-1725 (2010). It has been stated that, on a per molar basis, RNase 7 is one of the most potent human AMPs that has been described, with antibiotic concentrations ranging in the low micromolar range.

In the United States, urinary tract infections account for nearly seven million office visits, a million emergency department visits, and one hundred thousand hospitalizations every year. The cost of these infections is significant both in terms of lost time at work and costs of medical care. In the United States the direct cost of treatment is estimated at 1.6 billion USD yearly. Laboratory tests utilized in the diagnosis of urinary tract infections include urinalysis and urine cultures. The components of the urine analysis (UA) used to assist in the diagnosis of a UTI include the leukocyte esterase, nitrite and microscopy for white blood cells (WBCs) and bacteria. The sensitivity of these urinalysis parameters is so low that the risk of missing a urinary tract infection (UTI) is unacceptably high. Furthermore, false positives are common from skin contamination, non-infectious inflammatory conditions, and asymptomatic bacterial colonization leading to inappropriate use of antibiotics. While the UA cannot be used in lieu of urine cultures to document the presence of a UTI, in some cases it can assist in identifying patients that would benefit from the initiation of antibiotics while waiting for urine culture results. Pediatrics Guidelines for UTI diagnosis, Pediatrics. April 1999; 103(4 Pt 1):843-852. The diagnosis of UTI is confirmed or excluded by the results of a urine culture.

The number of colony forming units (CFU) required to grow on the culture media before a diagnosis of UTI is suspected varies depending on the gender of the patient and the method of urine collection. Bagged urine collection has a culture false positive rate of up to 99%. Further, urine culture results take 24-48 hours to be completed, and false positive results occur secondary to bacterial contamination or colonization. A more sensitive and specific rapid test for UTIs is needed. Such a test could, for example, aid clinicians in accurately diagnosing UTIs and determining whether to initiate antibiotics while urine culture results are pending.

### SUMMARY

A sensitive and rapid test useful for determining if a subject has an acute or chronic urinary tract infection is provided. In one aspect, the a method of identifying a subject having an acute urinary tract infection is described. The method includes the steps of providing a test sample of urine from the subject, determining the level of RNase 7 in the test sample, and comparing the level of RNase 7 in the test sample to the level of RNase 7 in a control. A higher level of RNase 7 in the test sample as compared to the level of RNase 7 in the control indicates that the subject has an acute urinary tract infection. In some embodiments, the method can also include the step of treating a subject identified as having an acute urinary tract infection with a therapeutic agent effective for treating an acute urinary tract infection, such as an antibiotic.

In another aspect, a method of identifying a subject having only a chronic urinary tract infection is described. The method includes providing a test sample of urine from the subject, determining the level of RNase 7 in the test sample, and comparing the level of RNase 7 in the test sample to the level of RNase 7 in a control. A lower level of RNase 7 in the test sample as compared to the level of RNase 7 in the control indicates that the subject has only a chronic urinary tract infection, and not an acute urinary tract infection.

### BRIEF DESCRIPTION OF THE FIGURES

The accompanying drawings, which are incorporated in and constitute a part of this specification, illustrate some embodiments disclosed herein, and together with the description, serve to explain principles of the disclosed embodiments.

FIG. 1 provides a bar graph showing abundant expression of RNase 7 in human kidney and bladder. Transcript levels quantified by real-time PCR in the kidney and bladder. Shown are the results of three independent samples. The mean transcript levels are shown with the SEM. RNase 7 expression was significantly greater in the bladder than the kidney (cortex, medulla, and pelvis) (p=0.003). RNase 7 expression was significantly greater in the renal pelvis than the cortex and medulla (p=0.0015).

FIG. 2 provides images showing that RNase 7 is expressed throughout the urothelium of the ureter and bladder. Immunohistochemistry demonstrates RNase 7 expression (arrows) throughout the urothelium of the human bladder (A) and ureter (C). Negative control bladder (B) and ureter (D). Magnification 40×.

FIG. 3 provides images showing that RNase 7 shows cell-specific expression throughout the human kidney. Immunohistochemistry demonstrates isolated cell specific expression (arrows) in the collecting duct of human kidney cortex (A) and medulla (C). Negative control cortex (B) and medulla (D). Magnification 20×.

FIG. 4 provides a graph showing that RNase 7 is present in urine. Urinary RNase 7 expression standardized to urine creatinine Shown are the ELISA results from 20 healthy controls.

FIG. 5 provides six graphs showing that RNase 7 contributes to impaired growth of E. coli and Pseudomonas in human urine. The antibacterial properties of RNase 7 were measured as changes in the turbidity of cultured human urine using the absorbance at 600 nm (OD₆₀₀). Human urine samples were inoculated at various urinary pH conditions with E. coli (Panels A-C) or Pseudomonas (Panels D-F) as shown by the solid black line. The open circles represent non-inoculated urine samples. Addition of irrelevant antibody to inoculated urine samples (dashed line) did not affect bacterial growth. Blocking of RNase 7 activity, with the addition of RNase 7 antibody (dashed line with diamonds), resulted in increased growth of all bacterial isolates.

FIG. 6 provides nine graphs showing that RNase 7 contributes to impaired bacterial growth in human urine. The antibacterial properties of RNase 7 were measured as changes in the turbidity of cultured human urine using the absorbance at 600 nm (OD₆₀₀). Human urine samples were inoculated with Klebsiella (Panels A-C), Enterococcus, (Panels D-F), or Proteus (Panels G-I) as shown by the solid black line. The open circles represent non-inoculated urine samples. Addition of irrelevant antibody to inoculated urine samples (dashed line) did not affect bacterial growth. Blocking of RNase 7, with the addition of RNase 7 antibody (dashed line with diamonds), resulted in increased growth of all bacterial isolates.

FIG. 7 provides a bar graph showing that RNase 7 expression levels are depressed by chronic infection, but are significantly raised during acute infection. Transcript levels quantified by real-time PCR in the kidney. Shown are the results of three independent samples.

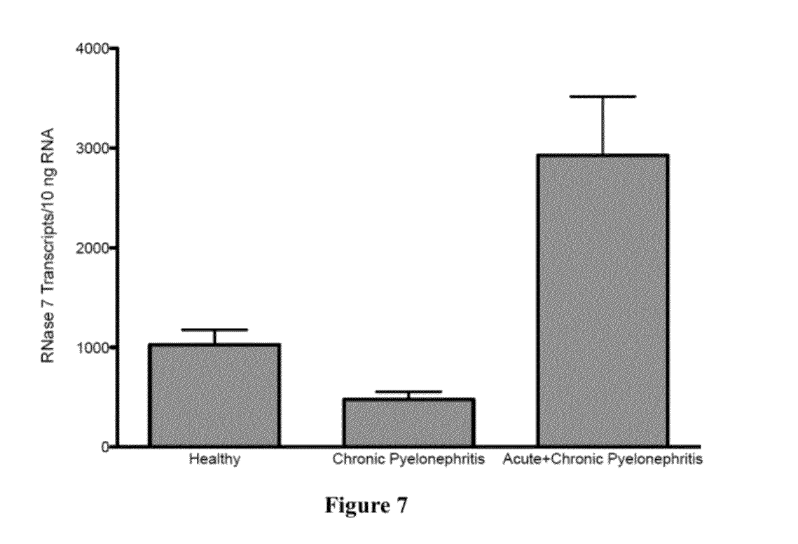

### DETAILED DESCRIPTION

Methods of identifying a subject having a urinary tract infection using RNase 7 are described herein. The methods include obtaining a test sample of urine from the subject, determining the level of RNase 7 in the test sample, and then comparing the level of RNase 7 in the test sample to the level of RNase 7 in a control. In one embodiment, a higher level of RNase 7 in the test sample indicates that the subject has an acute urinary tract infection. In another embodiment, a lower level of RNase 7 in the test sample indicates that the subject only has a chronic urinary tract infection.

The present embodiments will now be described by reference to some more detailed embodiments, with occasional reference to the accompanying drawings. However, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

The terms “individual,” “host,” “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Treatment of humans is of particular interest.

In some embodiments, the subject can be a subject who is at risk of having a urinary tract infection. A subject can be at risk of having a urinary tract infection for a variety of reasons. Being female is a risk factor, because women have a shorter urethra, which cuts down on the distance bacteria must travel to reach the bladder. Other risk factors include being sexually active, using certain types of birth control (e.g., diaphragms), undergoing menopause, having congenital urinary tract abnormalities, having blockages in the urinary tract (e.g., kidney stones or an enlarged prostate), having a suppressed immune system (e.g., as a result of diabetes, other diseases, or drug-based immunosuppression), or using a catheter to urinate.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a sample” includes a plurality of such samples.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

Methods of identifying a subject having a urinary tract infection are provided herein. The methods include providing a test sample of urine from the subject, determining the level of RNase 7 in the test sample, and comparing the level of RNase 7 in the test sample to the level of RNase 7 in a control. A higher level of RNase 7 in the test sample as compared to the level of RNase 7 in the control indicates that the subject has an acute urinary tract infection, whereas a lower level of RNase 7 in the test sample as compared to the level of RNase 7 in the control indicates that the subject has a chronic urinary tract infection. It is important to be able to distinguish between chronic and urinary tract infections, and prior art methods which simply determined whether a pathogen was present were unable to make this distinction.

Ribonuclease 7 (RNase 7), as defined herein, is a member of a class of proteins (nucleases) that catalyzes the degradation of RNA into smaller components. RNase 7 is 14.5 kDa protein that is part of the RNase A family of proteins. For a discussion of antimicrobial peptides including RNase 7, see Wiesner et al., Virulence. September-October; 1(5): 440-64 (2010). The amino acid sequence of RNase 7 is described by Harder J, Schroder J M., J.B.C.; 277: 46779-46784 (2002), the disclosure of which is incorporated herein by reference. RNase 7, as defined herein, includes proteins having the defined amino acid sequence, regardless of the presence of normal biochemical modifications of the protein, such as glycosylation, as well as other further modified proteins that retain RNase 7 activity.

### Urinary Tract Infection

A urinary tract infection, as defined herein, is an infection of any part of the urinary tract. The urinary tract includes the kidneys, the bladder, the urethra, and the ureter. Infection of these portions of the urinary tract typically result in a variety of symptoms. Infection of the kidneys (e.g., acute pyelonepthritis) can result in upper back and side pain, high fever, shaking and chills, nausea, and vomiting. Infection of the bladder (e.g., cystitis) can result in pelvic pressure, lower abdomen discomfort, frequent and painful urination, and blood in the urine. Infection of the urethra (e.g., urethritis) typically can be diagnosed based on a burning sensation associated with urination.

Urinary tract infections can be acute or chronic. An acute urinary tract infection is typically short term (i.e., less than one month) and of high intensity, whereas a chronic infection is a longer-term infection (i.e., lasting at least one month, and up to a number of years). A chronic infection and/or colonization is typically present when a patient has bacteria growing in their bladder but they do not have symptoms typically associated with a urinary tract infection. An acute infection is present when the patient has symptoms such as painful urination or fever. If an acute infection is present simultaneously with a chronic infection, the effects of the acute infection will dominate those of the chronic infection in terms of overall characterization of the infection, for at least the reason that a chronic infection typically shows few effects.

Urinary tract infections can also be asymptomatic. Asymptomatic bacteriuria is a colonization of a portion of the urinary tract by bacteria that does not display the symptoms typically seen for a urinary tract infection. The urine samples obtained from a subject with asymptomatic bacteriuria can look infected (as evaluated by dipstick, for example) and will result in bacterial growth if cultured. However, it is difficult to determine if this represents an early infection that can be treated briefly to avoid complications, or just bladder colonization with bacteria that does not represent a pathogenic infection and will likely not be cleared by treatment with antibiotics. Not all asymptomatic infections represent chronic infections. Some types of subjects will be asymptomatic as a result of a lack of inflammatory response due to immunosuppression (e.g., transplant patients) or have lack of sensation of symptoms as a result of, for example, spinal cord injuries and congenital spinal/neural tube defects.

A urinary tract infection is typically a bacterial infection. The bacteria can be gram-negative bacteria, or the bacterial can be gram-positive bacteria. For example, the bacteria can be one or more of E. coli, Pseudomonas, Enterococcus, Enterobacter, Klebsiella, or Proteus mirabilis. The majority (80-85%) of bacterial urinary tract infections are caused by E. coli. However, a urinary tract infection can also occur as a result of infection by pathogens other than bacteria. For example, urinary tract infections can also be caused by viruses and fungus. Examples of urinary viral infections include those by BK virus, cytomegalovirus (CMV) and Epstein-Barr virus (EBV). Fungal infection is commonly caused by infection by fungi of the genus Candida.

The amount of RNase 7 present can also indicate the location of infection. A higher level of RNase 7 indicates that an acute urinary tract infection is present. For example, a higher level of RNase 7 can be an RNase 7 level that is at least 10%, 25%, or 50% greater than that seen in the control. In addition, acute pyelonephritis (kidney infection) is a more severe condition than acute cystitis (bladder infection), and it is helpful to be able to distinguish between the two. Accordingly, in some embodiments of the invention, a level of RNase 7 in the test sample that is at least about 3 fold higher than the level of RNase 7 in the control indicates that the subject has acute pyelophritis.

### Methods for Measuring Levels of RNase 7

The levels of RNase 7 can be measured using any analytic method suitable for identifying proteins, including standard methods known in the art. Examples of methods that can be used to identify proteins include high performance liquid chromatography (HPLC), mass spectrometry (MS), gels (e.g., SDS-PAGE), and immunoassay. These methods can be used independently, or they can be combined. Examples of immunoassays include radioimmunoassay and enzyme-linked immunoassay (ELISA). In an immunoassay, the level of RNase 7 is determined using antibodies having an affinity for RNase 7. The degree of affinity can be varied to increase the specificity of binding to RNase 7 (i.e., high affinity), or to increase the ability to bind to closely related proteins (lower affinity). Immunoassays include a wide variety of variants, such as competitive, homogenous immunoassays, competitive, heterogeneous immunoassay, one-site, noncompetitive immunoassays, and two-site, noncompetitive immunoassays.

The analytic device used to measure the levels of RNase 7 can be either a portable or a stationary device. In addition to including equipment used for detecting the RNase 7, the analytic device can also include additional equipment to provide physical separation of analytes prior to analysis. For example, if the analyte detector is an immunoassay, it may also include an ion exchanger column chromatography to purify the proteins from urine before the specific detection of RNase 7 by immunoassay.

Once the levels of RNase 7 have been determined, they can be displayed in a variety of ways. For example, the levels of RNase 7 can be displayed graphically on a display as numeric values or proportional bars (i.e., a bar graph) or any other display method known to those skilled in the art. The graphic display can provide a visual representation of the amount of the RNase 7 in the urine samples being evaluated. In addition, in some embodiments, the analytic device can also be configured to display a comparison of the levels of RNase 7 in the subject's urine to a control value based on levels of RNase 7 in a comparable urine sample, urine samples from a reference cohort, or a standard numerical reference.

### Control Values

The levels of RNase 7 in the test sample obtained from the subject may compared to the RNase 7 level in a control. Generally, a control value is a concentration of an analyte (e.g., RNase 7) that represents a known or representative amount of an analyte in the control. A control can be a sample previously obtained from the subject when the subject was known to not have a urinary tract infection. Alternately, the control can be based on a control value derived from the levels of RNAse 7 in comparable samples obtained from a reference cohort. The reference cohort can be the general population, or in other embodiments, the reference cohort can be a select population of subjects (e.g., human subjects). In certain embodiments, the reference cohort is comprised of subjects who have not previously had any signs or symptoms indicating the presence of urinary tract infection. Symptoms indicating the presence of a urinary tract infection include a strong, persistent urge to urinate, a burning sensation when urinating, passing frequent, small amounts of urine, urine that appears cloudy, urine that appears bright pink or cola colored (a sign of blood in the urine), strong-smelling urine, and pelvic pain (in women) or rectal pain (in men). Preferably, the reference cohort includes individuals, who if examined by a medical professional would be characterized as being free of symptoms of urinary tract infection (i.e., asymptomatic).

The control value is preferably measured using the same units used to characterize the level of RNase 7 obtained from the subject. Thus, if the level of the RNase 7 is an absolute value such as the units of RNase 7 per ml of urine, the control value is also based upon the units of RNase 7 per ml of urine. Alternately, rather than stand-alone values, RNase 7 can also be measured in comparison to another substance present in the urine. For example, RNase 7 measurements can also be provided for both the test and controls normalized by comparison to creatinine levels. This type of control value can be considered an internal standard. Generally, an internal standard is a known amount of another compound that is already present or can be provided in a sample that can be measured along with the analyte to serve as a reference. The diagnostic methods described herein can also be carried out by determining the levels of RNase 7 in a urine sample and comparing them to the amount of an internal standard.

The control value can take a variety of forms. Control values of RNase 7 in urine samples obtained, such as mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference.

### Comparison of an RNase 7 Test Sample to an RNase 7 Control

Levels of RNase 7 in a sample from a subject may be compared to a single control value or to a range of control values. If the level of RNase 7 in the test sample is greater than the control value or exceeds or is in the upper range of the control values, the subject can be diagnosed as having an acute urinary tract infection. For example, an RNase 7 level that is at least 10%, 25%, or 50% greater than that seen in the control is a higher value the indicates that the subject has an acute urinary tract infection. In contrast, if the level of RNase 7 in the test sample is below the level of RNase 7 in the control, the subject can be characterized as having a chronic urinary tract infection. For example, an RNase 7 value that is about 25, 50, or 75% of the control indicates a lower level of RNase 7 that indicates a chronic infection. If the level of RNase 7 in the test sample is about the same as the level of RNase 7 in the control (e.g., with only about a 10% or less deviation from the control), the subject does not have a urinary tract infection. The extent of the difference between the level of RNase 7 in the test sample and the control can also useful for characterizing the degree and location of infection, and thereby determining which individuals would most greatly benefit from certain aggressive therapies.

The method of identifying a subject having a urinary tract infection can also include the further step of treating the subject with a therapeutic agent effective for treating a urinary tract infection. For example, it some embodiments, the therapeutic agent is an antibiotic. Examples of suitable antibiotics include trimethoprim-sulfamethoxazole, cephalosporins, nitrofurantoin, amoxicillin, Augmentin™, doxycycline, and fluoroquinolones. Pyelonephritis is treated more aggressively than a simple bladder infection using either a longer course of oral antibiotics or intravenous antibiotics. For a description of the various treatment methods for various types of urinary tract infection, see Orenstein et al., Am. Fam. Physician., 59(5): 1225-1234 (1999), the disclosure of which is incorporated by reference herein.

Urinary tract infections can also be treated with analgesics to relieve the burning pain and urgent need to urinate. For example, the local analgesic phenazopyridine hydrochloride (Pyridium®) can be used together with an antibiotic for treatment of a urinary tract infection.

In another embodiment, the present invention relates to kits that include reagents (e.g., RNase 7 specific antibodies) for assessing levels of RNase 7 in biological samples obtained from a subject. In certain embodiments, the kits also include printed materials such as instructions for practicing the present methods, or information useful for assessing whether the subject has a urinary tract infection. Examples of such information include, but are not limited to cut-off values, sensitivities at particular cut-off values, as well as other printed material for characterizing risk based upon the outcome of the assay. In some embodiments, such kits may also comprise control reagents, e.g., known amounts of RNase 7.

RNase 7 levels can be determined using samples from various tissues in the urinary tract. However, it is preferable to determine RNase 7 levels using a urine sample, since urine is readily available and can be obtained non-intrusively. The urine can be collected by any methodology including urine voided into a bag. The urine sample may be expressly obtained for use in the assays of this invention, or it may be obtained for another purpose which can then be sub-sampled for use in the assays of the present invention.

The urine sample may be pretreated as necessary by dilution in an appropriate buffer solution and concentrated or fractionated by any number of methods including but not limited to ultracentrifugation, fractionation by fast performance liquid chromatography (FPLC), or precipitation. Any of a number of standard aqueous buffer solutions at physiological pH, such as phosphate, Tris, or the like, can be used.

Evaluation of Therapeutic Agents for treating Urinary Tract Infection

Also provided are methods for evaluating the effect or effectiveness of therapeutic agents on individuals who have been diagnosed as having a urinary tract infection. Such therapeutic agents include, but are not limited to, antibiotics suitable for treating urinary tract infection such as sulfamethoxazole-trimethoprim, amoxicillin, nitrofurantoin, ampicillin, ciprofloxacin, and levofloxacin. The method includes determining levels of RNase 7 in a pre-treatment sample (e.g., urine sample) taken from the subject prior to therapy and determining the level of RNase 7 in a post-treatment sample taken from the subject during or following therapy. The pre-treatment sample and the post-treatment sample are then compared. For acute infection, this comparison will show a decrease in RNase 7 levels if treatment has been effective. For chronic infection, the comparison will shown an increase in RNase 7 levels if therapy has been effective.

A predetermined value can be based on the level of RNase 7 in a urine sample taken from a subject prior to administration of a therapeutic agent. In another embodiment, the predetermined value is based on the level of RNase 7 in comparable urine samples taken from control subjects that are apparently healthy, as defined herein.

The present invention is illustrated by the following example. It is to be understood that the particular examples, materials, amounts, and procedures are to be interpreted broadly in accordance with the scope and spirit of the invention as set forth herein.

### Example

### Characterization of the Expression and Relevance of RNase 7 in the Human Urinary Tract

Although the urinary tract is constantly challenged by microbial invasion, it remains free from microbial colonization. Little is known about how the urinary tract maintains sterility. Recent studies demonstrate the importance of antimicrobial peptides in protecting the urinary from infection. The inventors have characterized the expression and relevance of the antimicrobial peptide ribonuclease 7 (RNase 7) in the human kidney and urinary tract. Using RNA isolated from healthy human tissue, quantitative real-time PCR was performed to show basal RNASE7 expression in kidney and bladder tissue. Immunostaining localized RNase 7 to the urothelium of the bladder, ureter, and intercalated cells of the collecting tubules. In healthy control human urine samples, 5.6-20.0 μg RNase 7/mg creatinine was detected. Antibacterial neutralization assays showed that urinary RNase 7 has potent antimicrobial properties against gram-negative and gram-positive uropathogenic bacteria. The study demonstrates that RNase 7 is expressed in the human kidney and urinary tract and that it may play an important antimicrobial role in maintaining urinary tract sterility.

### Materials & Methods

Human Tissue and Urine Samples:

Human kidney and ureter tissue was obtained through the NCH Department of Pathology. Tissue samples were obtained from pediatric patients undergoing nephrectomy for Wilm's tumor. Tissue samples were free of microscopic signs of disease or inflammation. The tissue was preserved as neutral formalin-fixed paraffin-embedded sections and dissected into cortex, medulla, or renal pelvis before storage. Human bladder uroepithelium, snap-frozen in liquid nitrogen, was obtained from children undergoing ureteral re-implantation for reasons other than recurrent infection. Three bladder tissue specimens were obtained.

Urines samples (n=20) were obtained from healthy volunteers with no history of UTI. The urine samples were centrifuged to remove urine sediment and protease inhibitor cocktail was added (Thermo Scientific, Rockford, Ill., USA).

Ribonucleic Acid Isolation and Reverse Transcription:

Total RNA was isolated from frozen tissue using the Promega Total RNA Isolation System (Promega™, Madison, Wis., USA). For cDNA synthesis, 4-8 μg of total RNA was reverse transcribed with Superscript III reverse transcriptase using an oligo-thymidine primer according to the supplier's protocol (Invitrogen™, Carlsbad, Calif., USA). A single cDNA preparation from each specimen was used for the assay of all antimicrobial products tested.

Cloning of Gene Specific Plasmids for Standard Curves:

The cDNAs encoding RNase7 and GAPDH were cloned into a 4-Topo plasmid vector (Invitrogen™) according to the manufacturer's instructions. Plasmids were sequenced to confirm that the correct constructs were obtained. Serial dilutions of gene specific plasmids were quantitated (by both spectrophotometric absorbance at 260 nm and ethidium bromide staining agarose gel electrophoresis with DNA standards) and then used in real-time PCR to generate standard curves for each reaction.

Real-Time PCR:

Total cellular RNA was extracted from the collected specimens using SV Total RNA Isolation System (Promega™) according to the manufacturer's protocol. Real-time PCR was performed using single-stranded cDNA from human kidney and bladder tissue with specific oligonucleotide primer pairs using the 7500 Real-Time PCR System (Applied Biosystems™, Carlsbad, Calif., USA) equipped with a fluorescence detection monitor. PCR intron spanning primers were selected using previously published standards and sequences were confirmed using DNAstar® Laser Gene SeqBuilder. Koten et al., P1oS one; 4: e6424 (2009).

Briefly, cDNA corresponding to 10 ng RNA served as a template in a 25 μl reaction containing 75 nM of each primer, and 1× Light-Cycler-Fast Start DNA Master SYBR Green mix. The PCR conditions were: initial denaturation at 95° C. for 10 minutes, followed by 40 cycles with each cycle consisting of denaturation at 94° C. for 30 seconds, annealing at 62° C. for 30 seconds, and extension at 72° C. for 30 seconds. The cycle-to-cycle fluorescence emission was monitored at 530 nm and analyzed using 7500 Software V2.0.3 (Applied Biosystems™). Gene specific plasmid standards were included with every set of reactions. Absolute transcript levels are shown per 10 ng total RNA.

RNase 7 Antibodies:

Antibodies against RNase 7 were commercially purchased (Abcam, Cambridge, Mass., USA; Novus Biologicals, Littleton, Colo., USA; and Sigma-Aldrich, St. Louis, Mo., USA). According to the manufacturer, the Novus and Sigma antibodies do not cross-react with other proteins. The Abcam antibody does have some cross-reactivity between RNase 7 and RNase 8. However, prior studies indicate that human RNase 8 expression is limited to the placenta and absent in the kidney. Zhang et al., Nucleic Acids Res; 30: 1169-1175 (2002).

ELISA:

96-well flat-bottomed plates (Maxisorb, Nunc™, Rochester, N.Y., USA) were coated overnight at 4° C. with polyclonal antibody to RNase7 (3 μg/mL) (Abcam). After blocking with synthetic blocking buffer (Kem-En-Tec Diagnostics™, Denmark) for 2 hours at room temperature, standards and samples were added to the wells and incubated for 2 hours at room temperature. Serial dilutions of recombinant RNase 7 protein served as the standards (Novus Biologicals™). Following incubation with a different biotinylated (Lightning-Link Biotin Antibody Labeling Kit, Novus Biologicals™) polyclonal antibody to RNase7 (2 μg/ml) (Novus Biologicals™) for 2 hours at room temperature, streptavidin-horse radish peroxidase (Biolegend™, San Diego Calif., USA) was added for 30 minutes. After incubation with TMB substrate solution for 15 minutes (Kem-En-Tec Diagnostics™), the reaction was terminated with STOP solution (Cell Signaling Technology™, Danvers, Mass., USA) and read at a wavelength of 450 nm and with 570-nm background subtraction. The detection limit of the ELISA was 0.1 ng/ml.

Results from the ELISA assay were divided by urine creatinine to establish standardized urine RNase 7-to-creatinine ratios (μg/mg) to account for urine dilution. Urine creatinine concentrations were determined using the Oxford Biomedical Research™ creatinine microplate assay (Rochester Hills, Mich., USA).

Immunoblot Analysis:

Urinary proteins were extracted from human urine samples using the Proteospin™ Urine Protein Concentration Micro Kit according to manufacturer's instructions (Norgen Biotek Corporation™, Thorold, ON, Canada). The urine protein samples were mixed with Laemmli sample buffer and incubated at 95° C. for 5 minutes. The samples were loaded onto 18% SDS gradient gels and subjected to electrophoresis (applied constant voltage of 100 V). After the peptide/protein separation, the material in the gel was transferred to nitrocellulose by application of 100 V for 90 minutes. The membranes were blocked in 2% fat-free milk and incubated with the rabbit polyclonal RNase 7 antibody (Abcam™, Cambridge, Mass., USA) in PBS with 2% fat-free milk overnight at 4° C. Incubation with the secondary antibody, a monkey horseradish peroxidase-conjugated anti-rabbit IgG diluted 1:8000 in PBS with 5% fat-free milk, for 1 hour at room temperature. The proteins and peptides were visualized using an ECL detection system and chemiluminescence film according to the manufacturer's instructions (BioExpress™, Kaysville, Utah, USA).

Immunohistochemistry:

Immunohistochemistry was performed on human kidney specimens to evaluate RNase 7 expression at the cellular level. Following deparaffinization and rehydration, antigen retrieval was performed in a pressure cooker for 20 minutes using 0.01 mol/L citrate buffer (pH 6.0). This step was followed by a biotin block and a serum-free protein block (Superblock™, ScyTek Laboratories, Logan, Utah, USA). The slides were incubated overnight at 4° C. with polyclonal rabbit RNase 7 antibody (Sigma-Aldrich) diluted 1:50 in PBS containing 3% fetal bovine serum followed by anti-polyvalent biotinylated antibody (anti-mouse, rat, rabbit, guinea pig) and UltraTek Streptavidin/HRP (ScyTek Laboratories™).

Sections were developed using 0.1% diaminobenzidine tetrachloride (Arcos Organics™, Geel, Belgium) with 0.02% hydrogen peroxide and counterstained with hematoxylin. Negative controls sections were incubated with non-immune serum in place of RNase7 antibody.

Immunofluorescence:

Double-labeled immunofluorescence was performed to help localize RNase 7 expression in the kidney. Sections were double-labeled for principal cells with goat polyclonal anti-human AQP-2 antibody (Santa Cruz Biotechnology™, Santa Cruz, Calif., USA).

Sections were double-labeled for intercalated cells with mouse IgG₁ monoclonal anti-human AE-1 antibody (gift from M. Jennings) or mouse IgG₁ monoclonal anti-human pendrin antibody (Medical & Biological Laboratories™, Naka-ku Nagoya, Japan). α-intercalated cells show basolateral AE-1 expression and β-intercalated cells show apical pendrin expression. Wall S M., Current opinion in nephrology and hypertension; 14: 480-484 (2005). Rhodamine donkey polyclonal anti-goat (Jackson ImmunoResearch Laboratories™, West Grove, Pa., USA), rhodamine goat anti-mouse (Jackson ImmunoResearch Laboratories™), and FIT-C donkey polyclonal anti-rabbit (Santa. Cruz) served as the secondary antibodies.

All sections were prepared as outlined above. They were incubated with a mixture of antisera against RNase 7 (1:50) (Sigma-Aldrich) and antisera against AE-1 (1:50), pendrin (1:50), or AQP-2 (1:200) at room temperature for 90 minutes. The secondary antibody was applied for 1 hour at room temperature and the sections were mounted using mounting media with DAPI. Non-immune serum was used as a negative control.

The slides were examined with a Leica DM4000B microscope and digitally photographed using the 100× objective and Spot RT camera/software (Diagnostic Instruments™, Sterling Heights, Mich., USA). The final images were processed with Adobe Photoshop software (Adobe Systems™, San Jose, Calif., USA).

Antimicrobial Neutralization Assay:

RNase 7 antimicrobial activity against uropathogenic Escherichia coli (UTI-89), Pseudomonas aeruginosa (PEDUTI-61), Enterococcus (PEDUTI-39), Klebsiella (PEDUTI-65), and Proteus mirabilis (PEDUTI-44) was evaluated in human urine. These bacterial strains were isolated from positive urine cultures of patients at NCH. In brief, the bacteria were cultured at 37° C. overnight in Luria-Bertani broth to saturation. 1 μL of bacteria was added to 100 μL a healthy individual in a 96-well flat bottom plate (Thermo Scientific, Nunc™, Worcester, Mass., USA). 10 μg of anti-RNase 7 antibody (Novus) or equivalent concentrations of irrelevant antibody (derived from preimmune goat serum) were added to each well. Koten et al., P1oS one; 4: e6424 (2009).

Bacterial growth was monitored using a Synergy HT multi-mode microplate reader (BioTek Instruments™, Winooski, Vt., USA) at a final volume of 101 μL. The turbidity of the culture was measured and recorded at t=0 and every ten minutes thereafter for 10 hours using the absorbance at 600 nm (O.D.₆₀₀). Sterility of urine was validated by incubation in the absence of bacterial inoculation. The assays were performed at a urinary pH of 5.0, 7.0, and 9.0. The urinary pH was adjusted by the titration of 0.1 N hydrochloric acid or sodium hydroxide.

### Results

RNase 7 is slightly upregulated during simple bladder infection (cystitis) and massively upregulated during kidney infection (pyelonephritis). A 3-fold increase of tissue expression was seen during pyelonephritis in the kidney compared to sterile conditions. Real time PCR data shows ˜3000 copies/10 ng RNA in infection versus 1000 copies/10 ng RNA during normal sterile conditions. Expansion of expression from the collecting ducts (only localization during sterile conditions) was seen to include the collecting ducts and proximal tubules of the kidney during infection using immunohistochemistry (IHC) and immunofluorescence (IF). ELISA data on a sterile patients 5.2-13 μg RNase 7/mg creatinine (Cr). Simple cystitis had a concentration of 5-10 μg/mg Cr. Pyelonephritis had 19.4-27 μg/mg Cr.

Researchers have been searching for years for a biomarker to differentiate between the two (procalcitonin is the most recent attempt, but not a reliable marker). Differentiating between pyelonephritis and cystitis is difficult, especially in younger patients. Early identification and treatment of pyelonephritis is critical in initiating appropriate therapy (inpatient management, IV antibiotics) to prevent renal scarring and urosepsis. Furthermore, identification of pyelonephritis warrants follow-up studies (VCUG to identify underlying urinary tract anomalies, DMSA renal scan for renal scarring).

RNase 7 has been shown to be upregulated in the skin during infection. The expression levels in the kidney and urinary tract as of now are unknown, and tissue response to infection is unknown. RNase 7 mRNA expression in the kidney is upregulated 3-fold during kidney infection (pyelonephritis). In the urine, levels are significantly higher during kidney infection compared to simple cystitis or sterile conditions. The data provided herein demonstrate that RNase 7 urine protein-to-creatinine ratio is a rapid, sensitive, and inexpensive biomarker for pyelonephritis. This test will further inform clinicians on stratifying urinary tract infections for work-up and management.

Human Kidney and Bladder Tissue Express High Levels of RNASE7 mRNA.

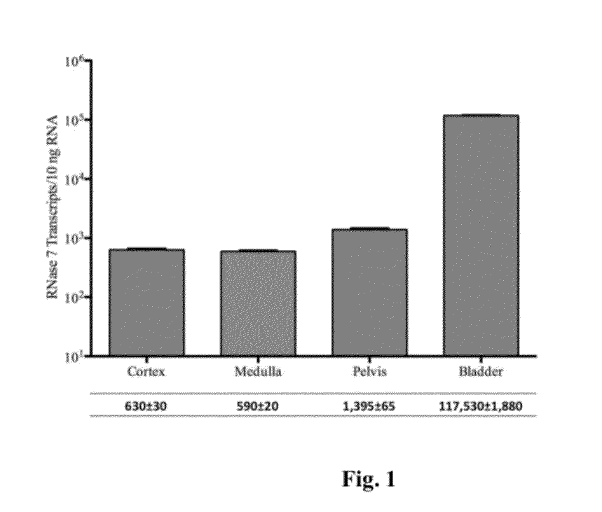

In the bladder, mean RNASE7 expression was 117,530±1,880 transcripts per 10 ng RNA. RNASE7 expression was significantly greater in the bladder than within the kidney (p=0.0003). In the kidney, RNASE7 expression was analyzed separately in the cortex, medulla, and pelvis. All tissue sections tested expressed RNASE7 (FIG. 1). RNASE7 expression was greatest in the renal pelvis compared to other parts of the kidney with a mean RNASE7 expression of 1,395±65 transcripts per 10 ng RNA. RNASE7 expression was significantly greater in the renal pelvis than the renal cortex and medulla (p=0.0015).

RNase 7 is Expressed Throughout the Human Kidney and Urinary Tract.

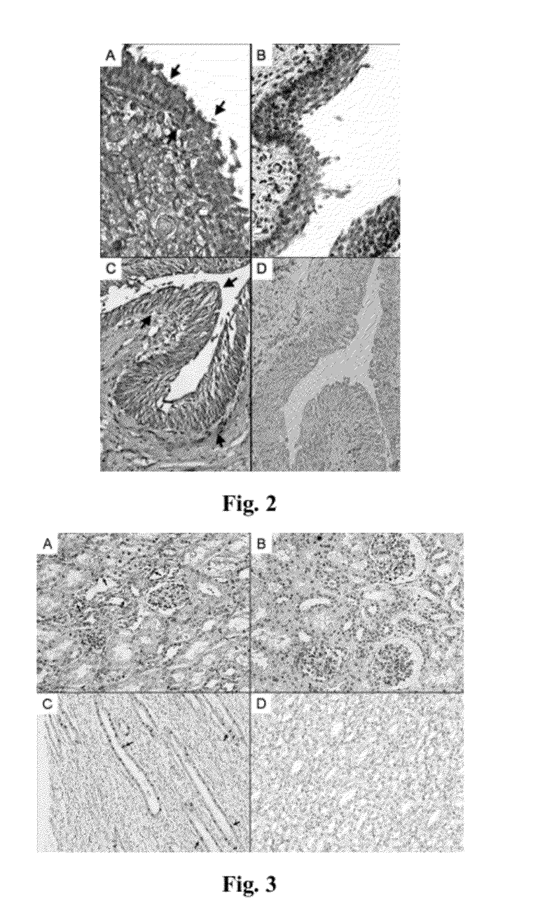

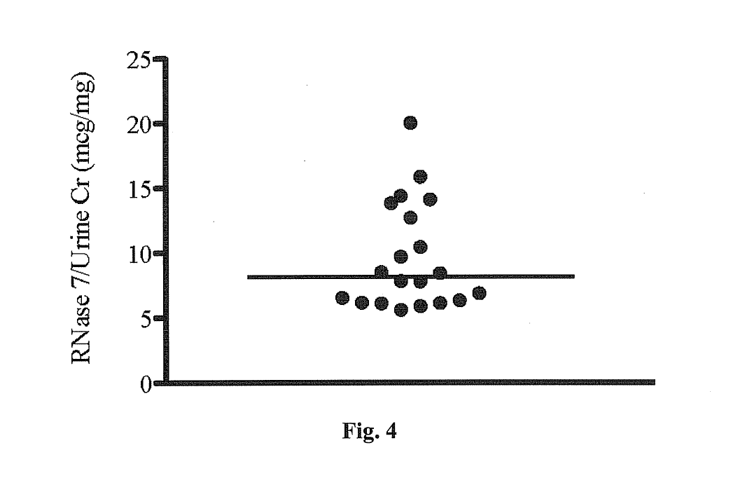

To investigate the distribution of RNase 7 in healthy human kidney, ureter, and bladder tissue, immunohistochemistry and immunofluorescence were performed using antibodies directed against RNase 7. RNase 7 immunoreactivity was present throughout the urothelium of the ureter and bladder of all investigated specimens (n=4) (FIG. 2). RNase 7 was also expressed in the renal cortex and renal medulla of all specimens (FIG. 3). RNase 7 showed cell-specific expression in the cortical and medullary collecting tubules. The glomeruli, proximal tubules, and loops of Henle did not show RNase 7 staining Negative controls showed no RNase 7 immunoreactivity (FIGS. 2 and 3).

RNase 7 is expressed in a specific subset of cells in the human collecting duct. Expression of RNase 7 in individual cells types of the collecting duct was performed using markers for principal cells (aquaporin-2 (AQP-2)), α-intercalated cells (anion exchanger-1 (AE-1)), and β-intercalated cells (pendrin) (data not shown). Human kidney was labeled for RNase 7, nuclei and cell type markers. Cell type markers consisted of AQP-2 for principal cells, AE-1 for α-intercalated cells, and pendrin for β-intercalated cells. Isolated cells positive for RNase7 were identified in the collecting tubule. Principal cells were negative for RNase 7. RNase 7 was expressed by α-intercalated cells (red basolateral AE-1 staining) RNase 7 and pendrin co-localized in β-intercalated cells to demonstrate apical yellow staining α- and β-intercalated cells expressed RNase 7, but principal cells did not. Occasionally, pendrin positive and AE-1 positive cells did not demonstrate RNase 7 expression. Negative controls showed no RNase 7 expression. In addition, only nephron segments positive for AQP-2 contain cells positive for RNase 7, indicating that RNase 7 staining in the human kidney is limited to the collecting duct. Within the collecting duct, RNase 7 staining is limited to cells without AQP-2 staining.

RNase 7 Exists in Human Urine in Measurable Titers.

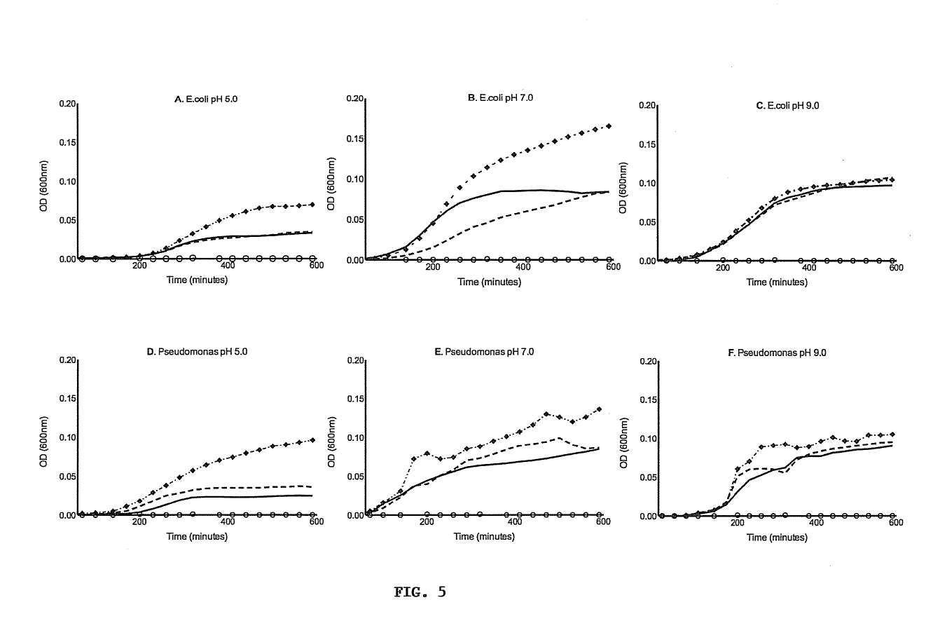

Immunoblot analysis of cationic RNase 7 protein extracted from urine specimens identified an immunoreactive peptide that migrated to 14.5 kDa (data not shown). ELISA assay demonstrated that RNase 7, normalized to urine creatinine, was present in all urine samples with concentrations ranging from 5.60-20.0±0.92 μg/mg creatinine (Cr) (235 μg/L-3467.2 μg/L), which corresponds to 0.17-0.3 μM (FIG. 4).

RNase 7 Displays Antibacterial Properties in Human Urine.

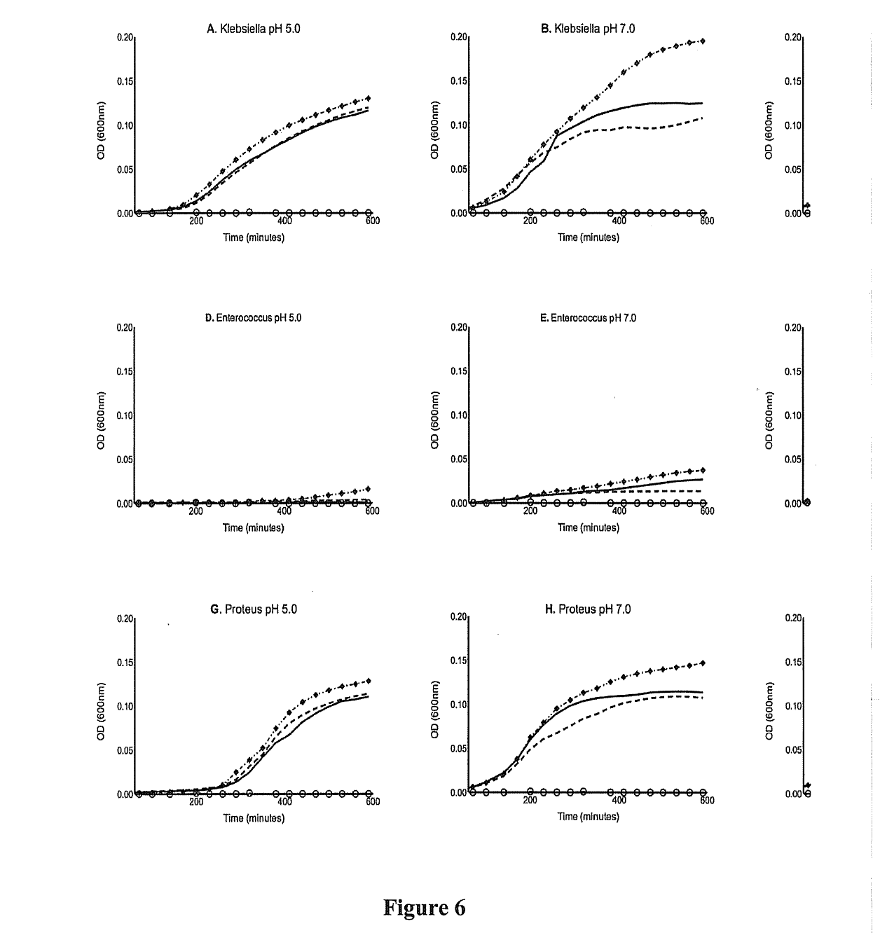

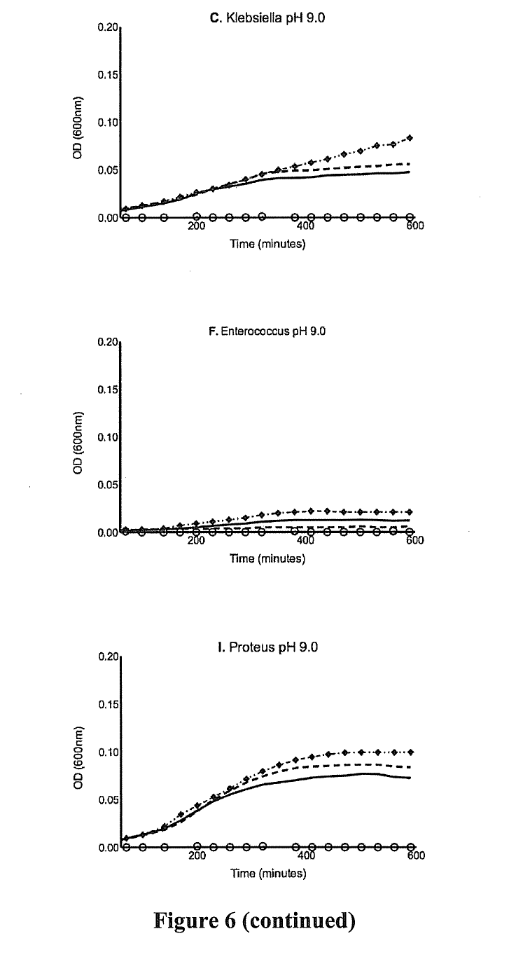

To determine if RNase 7 has antibacterial effects in human urine, urine samples from healthy individuals were inoculated with either uropathogenic E. coli, Pseudomonas, Enterococcus, Klebsiella, or Proteus mirabilis (FIG. 5 and FIG. 6). Next, the inventors analyzed whether the addition of antibodies directed against RNase 7 would neutralize the antimicrobial activity of RNase 7 and lead to enhanced bacterial growth. RNase 7 antibodies inhibited the action of RNase 7 as bacterial growth of gram-negative bacteria and Proteus significantly increased (FIG. 5 and FIG. 6). The presence of equivalent concentrations of irrelevant antibodies (derived from goat pre-immune serum) had no effect on bacterial growth.

Because the pH of human urine is highly variable, this approach was used to determine the effective pH range for the antimicrobial effects of RNase 7 in urine. Urine samples from healthy individuals were buffered to pH 5.0, 7.0 or 9.0. Bacterial growth was monitored (OD 600 nm) in the presence or absence of antibodies directed against RNase 7. Significant bacterial growth was observed at all pH values tested, although growth was impeded in alkaline conditions (FIG. 5 and FIG. 6). Enterococcus demonstrated very little growth under all conditions (FIG. 6). These results are consistent with prior reports on the influence of pH on bacterial growth. Asscher et al. Lancet; 2: 1037-1041 (1966).

The application of antibodies directed against RNase 7 resulted in increased bacterial growth under most urinary pH values tested. However, this phenomenon was not uniform under all conditions (FIG. 5 and FIG. 6).

RNase 7 expression levels were also evaluated in kidneys for subjects having chronic pyelonephritis or chronic pyelonephritis as well as acute pyelonephritis. The data were obtained using multiple samples and real time PCR as has already described herein. The results show a 3-fold increase during an acute infection (together with a chronic infection) and a ˜50% decrease during chronic infection. This indicates that chronic infection; i.e., an asymptomatic bacteriuria/colonization that represents a chronic, smoldering, low level infection is discernable from acute infection using RNase 7 by comparing it to a “baseline” value in a subject who may be at risk of having a urinary tract infection.

TABLE I
Comparison of RNase 7 expression in chronic and acute infection

| | | Chronic | |
|---|---|---|---|
| | Healthy | Pyelo | Acute + Chronic Pyelo |
| Number of values | 4 | 4 | 6 |
| Minimum | 725.1 | 322 | 659.4 |
| 25% Percentile | 757.3 | 334.2 | 1703 |
| Median | 997.7 | 470.1 | 3016 |
| 75% Percentile | 1330 | 631.4 | 4286 |
| Maximum | 1393 | 652 | 4662 |
| Mean | 1028 | 478.5 | 2927 |
| Std. Deviation | 299 | 157.6 | 1446 |
| Std. Error | 149.5 | 78.79 | 590.5 |
| Lower 95% CI of mean | 552.7 | 227.8 | 1409 |
| Upper 95% CI of mean | 1504 | 729.3 | 4445 |
| Sum | 4114 | 1914 | 17565 |

Units are Transcripts RNase 7/10 ng RNA

### Discussion

Prior studies have demonstrated that RNase 7 is an important AMP in skin, hair follicles, and the oral cavity. Koten et al. P1oS one; 4: e6424 (2009); Eberhard et al., Oral microbiology and immunology; 23: 21-28 (2008); Reithmayer et al. Br J Dermatol; 161: 78-89 (2009). In this study, the inventors demonstrate that RNase 7 is a novel AMP expressed in the human urinary tract. The results demonstrate that RNase 7 is constitutively expressed in the mature human kidney, ureter, and bladder. Using quantitative real-time PCR and ELISA, the inventors demonstrate that RNase 7 activity is greatest in the bladder and renal medulla. Specifically, immunohistochemical labeling demonstrates that RNase 7 is expressed throughout the urothelium of the lower urinary tract and the intercalated cells of the collecting tubules. These results suggest that intercalated cells have a novel role in innate immunity. Finally, high concentrations of RNase 7 were identified in the urine and it was demonstrated that RNase 7 has urinary antimicrobial activity against a variety of uropathogenic bacteria.

The quantitative real-time PCR results demonstrate that RNASE7 is expressed at high levels throughout the urinary tract. The basal uroepithelial expression of RNASE7 is greater than the expression of previously described urinary tract AMPs like cathelicidin, human β-defensin 1 (HBD-1), and HBD-2. Chromek et al., Nature medicine; 12: 636-641 (2006); Lehmann et al., BMC infectious diseases; 2: 20 (2002). Furthermore, the results indicate that renal RNASE7 expression is comparable to RNASE7 expression in keratinocytes. When comparing RNASE7 expression in the bladder to RNASE7 expression in the skin, the results demonstrate that the bladder expresses nearly 100-times more RNASE7 than primary keratinocytes. High expression levels may be required in the bladder to produce bactericidal titers of RNase 7 because it is constantly excreted into and/or diluted by urine.

The quantitative real-time PCR results also demonstrate that RNASE7 expression increased from the upper urinary tract to the lower urinary tract—following the flow of the urinary stream. Similarly, immunostaining demonstrated that RNase 7 expression is more homogeneous throughout the lower urinary tract. IHC demonstrated cell-specific RNase 7 expression in a minority of isolated cells in the cortical and medullary collecting tubules before becoming more uniform throughout the uroepithelium of the ureter and bladder. RNase 7 was not expressed in the glomeruli, proximal tubules, loops of Henle, or interstitium. This expression pattern differs from the expression of other AMPs that have been described in the kidney. For example, hBD-1, hBD-2, and cathelicidin do not show cell-specific expression, and they have limited or no intracellular expression. Harder et al., Nature; 387: 861 (1997); Valore et al., The Journal of clinical investigation; 101: 1633-1642 (1998).

Because RNase 7 protein expression is limited to a minority of cells in the renal cortex and medulla, only a small percentage of cells account for the relatively high RNASE7 expression levels, especially when compared to more homogenous epithelial organs like the epidermis. Overall, both real-time PCR and IHC indicate that RNase 7 expression is present in locations where microbial exposure occurs most frequently. A similar expression pattern has been described in the epidermis and in hair follicles. In the skin, RNase 7 expression is greatest in the uppermost epidermal layers, where microbial insult most likely occurs. Likewise, in hair follicles RNase 7 expression is greatest in the outer root sheath.

The results indicate that α- and β-intercalated cells constitutively express RNase 7. Historically, intercalated cells have been shown to play important roles in the regulation of acid/base homeostasis. Intercalated cells account for ⅓ of the cells within the cortical and medullary collecting ducts. Wall, S. M., Current opinion in nephrology and hypertension; 14: 480-484 (2005). Given their physiologic position in the collecting duct, intercalated cells are ideally positioned to defend the kidney from ascending urinary tract infections as they are one of the initial cell types encountered by ascending microbes before they infiltrate the renal parenchyma. The identification of RNase 7 in both subtypes of intercalated cells defines a new role for these cells, indicating that they are critical for the production and secretion of AMPs into the urine.

The ELISA results indicate that RNase 7 is secreted into the urine. Given the size of RNase 7 (14.5 kDa), it is possible that some urinary RNase 7 peptides originate, at least in part, from plasma filtrate. However, there is little evidence suggesting that RNase 7 persists in the plasma. Zhang et al., Nucleic acids research; 31: 602-607 (2003). Additionally, to persist in the urine, RNase 7 would need to escape the efficient peptide absorption mechanisms in the proximal tubule. Carone et al., J Lab Clin Med; 100:1-14 (1982); Christensen et al., Current opinion in nephrology and hypertension; 6: 20-27 (1997). Finally, the urine samples underwent centrifugation before processes, removing cellular sources of RNase 7. This finding suggests the predominant source of urinary RNase 7 most likely originates from local production by intercalated cells of the distal nephron and the urothelium of the bladder and ureters.

In the inventors' study of 20 healthy individuals, constitutive urinary RNase 7 protein concentration was between 5.6-20.0 μg/mg Cr (0.17 μM-0.3 μM). When comparing urinary RNase 7 concentrations to the concentrations of other urinary AMPs (i.e. cathelicidin, hBD-1, and hBD-2), RNase 7 concentrations are much greater. hBD-2 is not constitutively secreted in the urine while median urinary concentrations of cathelicidin and hBD-1 are 1.6×10⁻⁵ μM and 2.5×10⁻⁵ μM, respectively. Constitutive RNase 7 expression at these concentrations shows potent antimicrobial activity against several pathogenic microbes, including Pseudomonas aeruginosa, Staphylococcus aureus, and vancomycin-resistant E. faecium. Lin et al., J.B.C., 285: 8985-8994 (2010); Simanski et al., J Invest Dermatol., 130: 2836-8 (2010).

As observed in other systems, RNase 7 demonstrates antimicrobial activity in the urine. When the antimicrobial activity of RNase 7 was inhibited with antibodies directed against RNase 7, bacterial growth of E. coli, Pseudomonas, Klebsiella, and Proteus significantly increased. This effect was observed at all urinary pH values, but was less pronounced under alkaline urine conditions. This phenomenon may be secondary to decreased activity of RNase 7 at a higher pH, or it may reflect differential expression of microbial proteins that are important for RNase 7 antimicrobial activity. The effect of urine pH on RNase 7 activity and bacterial gene expression are currently under investigation. Overall, these data suggest that RNase 7 is involved in maintaining urine sterility, which is consistent with the proposed role of RNase 7 in innate immunity and antimicrobial defense. Boix et al., Mol Biosyst; 3: 317-335 (2007).

In conclusion, this is the first study to identify and evaluate RNase 7 in the human kidney and urinary tract. The results suggest that RNase 7 is an epithelial-derived AMP that plays an important role in the innate immunity of the human uroepithelium. The inventors demonstrate that RNase 7 contributes to host defense against gram-negative and gram-positive bacteria.

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while theories may be presented describing operation of the invention, the inventors are not bound by theories described herein. The invention is not limited to the exact details shown and described, for variations obvious to one skilled in the art will be included within the invention defined by the claims.

What is claimed is:

1. A method of identifying a subject having an acute urinary tract infection, comprising:
providing a test sample of urine from the subject;
determining the level of RNase 7 in the test sample;
comparing the level of RNase 7 in the test sample to the level of RNase 7 in a control;
wherein a higher level of RNase 7 in the test sample as compared to the level of RNase 7 in the control indicates that the subject has an acute urinary tract infection.

2. The method of claim 1, wherein the subject is human.

3. The method of claim 1, wherein the infection is a bladder infection.

4. The method of claim 1, wherein the infection is a kidney infection.

5. The method of claim 4, wherein the level of RNase 7 in the test sample is at least about 3 fold higher than the level in the control.

6. The method of claim 1, wherein the infection is a bacterial infection.

7. The method of claim 1, wherein the level of RNase 7 in the test sample is determined using an immunoassay with antibodies having an affinity for RNase 7.

8. The method of claim 1, wherein the control is a urine sample previously obtained from the subject.

9. The method of claim 1, wherein the control is the RNase 7 to creatinine ratio present in a subject without a urinary tract infection.

10. The method of claim 1, further comprising the step of treating a subject identified as having an acute urinary tract infection with a therapeutic agent effective for treating an acute urinary tract infection.

11. The method of claim 10, wherein the therapeutic agent is an antibiotic.

12. A method of identifying a subject having only a chronic urinary tract infection, comprising:
providing a test sample of urine from the subject;
determining the level of RNase 7 in the test sample;
comparing the level of RNase 7 in the test sample to the level of RNase 7 in a control;
wherein a lower level of RNase 7 in the test sample as compared to the level of RNase 7 in the control indicates that the subject has only a chronic urinary tract infection.

13. The method of claim 12, wherein the subject is human.

14. The method of claim 12, wherein the infection is a bladder infection.

15. The method of claim 12, wherein the infection is a kidney infection.

16. The method of claim 12, wherein the level of RNase 7 in the test sample is at least about 50% lower than the level in the control.

17. The method of claim 12, wherein the infection is a bacterial infection.

18. The method of claim 12, wherein the level of RNase 7 in the test sample is determined using an immunoassay with antibodies having an affinity for RNase 7.

19. The method of claim 12, wherein the control is a urine sample previously obtained from the subject.

20. The method of claim 12, wherein the control is the RNase 7 to creatinine ratio present in a subject without a urinary tract infection.
